(19) United States
(12) Patent Application Publication
(10) Pub. No.: US 2025/0064667 A1
(43) Pub. Date: Feb. 27, 2025

(54) BEAUTY HEAD AND BEAUTY APPARATUS

(71) Applicant: JUEQING HE, GUANGZHOU (CN)

(72) Inventor: JUEQING HE, GUANGZHOU (CN)

(21) Appl. No.: 18/625,161

(22) Filed: Apr. 2, 2024

(30) Foreign Application Priority Data
Aug. 22, 2023 (CN) 202311066418.8

(51) Int. Cl. A61H 7/00; A61M 35/00; A61N 1/36

(52) U.S. Cl. CPC A61H 7/005; A61M 35/003; A61N 1/3603

(57) ABSTRACT

A beauty head and a beauty apparatus include a head body, a first physiotherapy piece, and at least one ejecting head. The first physiotherapy piece is arranged on a beauty physiotherapy side of the head body. A compression cavity is defined between the first physiotherapy piece and the head body. When the first physiotherapy piece is compressed, the compression cavity discharges air therein. When the compression cavity resets, liquid in the liquid storage cavity is sucked into the compression cavity through a conveying channel communicated a liquid storage cavity. When the first physiotherapy piece is compressed again, the compression cavity is compressed, and a check valve in the conveying channel prevents the liquid from flowing into the liquid storage cavity. Therefore, the liquid is only allowed to be ejected out through the at least one ejecting head communicated with the compression cavity.

### TECHNICAL FIELD

The present disclosure relates to a technical field of beauty equipment, and in particular to a beauty head and a beauty apparatus.

### BACKGROUND

With development of technology, more and more people choose to use home beauty apparatuses for beauty care, and functions of the beauty apparatuses are more and more diverse. For instance, an electric pulse massage beauty apparatus is a device that contacts a human skin through electrode slices, so as to stimulate the human skin and simulate physical massage, tapping, compressing, and other effects. Moreover, a spectrum beauty apparatus is a device that uses different wavelengths of spectrum to stimulate energy in skin cells, accelerate skin blood circulation, stimulate fibroblast tissue to produce collagen, repair aging skin and acne, fade spots, and improve skin color.

However, when using a conventional beauty apparatus, it is generally necessary to use the conventional beauty apparatus with cosmetics or beauty lotions. Generally, the cosmetics or the beauty lotions are applied to the human skin first, and then the conventional beauty apparatus is operated. When it needs to add the cosmetics or the beauty lotion on the human skin, the beauty apparatus needs to be stopped first, which is inconvenient to use and affects a use experience.

### SUMMARY

Based on this, in order to solve above problems, it is necessary to provide a beauty head and a beauty apparatus that are convenient to use.

The beauty head comprises a head body, a first physiotherapy piece, and at least one ejecting head. A liquid storage cavity is defined in the head body. One side of the head body is a beauty physiotherapy side. The first physiotherapy piece is arranged on the beauty physiotherapy side of the head body. A compression cavity is defined between the first physiotherapy piece and the head body. The first physiotherapy piece is movable relative to the head body to compress or reset the compression cavity. The compression cavity is communicated with the liquid storage cavity through a conveying channel. A check valve is arranged in the conveying channel. The check valve is configured to control one-way flow of liquid from the liquid storage cavity to the compression cavity. The at least one ejecting head is arranged on the beauty physiotherapy side of the head body. The compression cavity is communicated with an ejecting space defined in the at least one ejecting head.

In one embodiment, the at least one ejecting head defines an ejecting outlet communicated with the ejecting space. The first physiotherapy piece moves toward the head body to compress the compression cavity, so that the ejecting outlet is exposed outside the first physiotherapy piece. The first physiotherapy piece is reset to reset the compression cavity to cover the ejecting outlet.

In one embodiment, one side of the at least one ejecting head facing the first physiotherapy piece is an inclined ejecting surface. The inclined ejecting surface of the at least one ejecting head is inclined toward a direction away from the first physiotherapy piece along a direction in which the first physiotherapy piece compresses the compression cavity. The ejecting outlet is defined on the inclined ejecting surface of the at least one ejecting head.

In one embodiment, the first physiotherapy piece is of a disc-shaped structure. The first physiotherapy piece is snapped on the head body. ejecting heads are provided. The ejecting heads are arranged around an axis of the first physiotherapy piece at intervals. The ejecting space of each of the ejecting heads are communicated with the compression cavity.

In one embodiment, the beauty head further comprises a first electric control piece. The first physiotherapy piece is electrically connected to the first electric control piece. The first electric control piece is configured to control the first physiotherapy piece to generate micro-current. A compression groove is defined on the beauty physiotherapy side of the head body. The first physiotherapy piece is mounted on the head body to enable the first physiotherapy piece to cover the compression groove to form the compression cavity. An inner surface of the first physiotherapy piece is configured as an inner wall of the compression cavity.

In one embodiment, the beauty head further comprises elastic reset pieces. The elastic reset pieces are arranged on the inner side of the first physiotherapy piece, and the elastic reset pieces reset to provide elastic forces for the first physiotherapy piece to reset the compression cavity. The first physiotherapy piece is electrically connected to the first electric control piece through the elastic reset pieces.

In one embodiment, the beauty head further comprises a heating piece. The heating piece is attached to the first physiotherapy piece. The heating piece is electrically connected to the first electric control piece. The first electric control piece is configured to control a heating temperature of the heating piece.

In one embodiment, the beauty head further comprises a light-transmitting piece and a light source. The light source is arranged on the first electric control piece. The light-transmitting piece is arranged on the beauty physiotherapy side of the head body and faces the light source. An outer surface of the light-transmitting piece is concave in a direction facing the compression cavity relative to the outer surface of the first physiotherapy piece. The light-transmitting piece is an annular piece. The first physiotherapy piece is located in an inner ring of the light-transmitting piece. A limiting space is defined on a side of the light-transmitting piece facing the first physiotherapy piece. The at least one ejecting head is arranged in the limiting space.

Before using the beauty head, beauty liquid or cosmetics applied on a skin of a human body need to be added to the liquid storage cavity. When in use, the first physiotherapy piece located on the beauty physiotherapy side of the head body is attached to the skin, and the first physiotherapy piece performs physiotherapy on the skin. When the beauty liquid or the cosmetics are needed, the beauty head is pressed so that the first physiotherapy piece directly presses against the skin. Under a reaction of the skin, the first physiotherapy piece is compressed, so that the compression cavity defined between the first physiotherapy piece and the head body is also compressed to discharge air in the compression cavity. When the first physiotherapy piece resets, a volume of the compression cavity increases. Since the compression cavity and the liquid storage cavity are communicated through the conveying channel, the cosmetics or the beauty liquid in the liquid storage cavity is sucked into the compression cavity when the volume of the compression cavity increases. When the first physiotherapy piece is compressed again, the compression cavity is compressed, and the volume of the compression cavity reduces. The check valve arranged in the conveying channel prevents the beauty liquid or the cosmetics from flowing into the liquid storage cavity. Therefore, the beauty liquid or the cosmetics are only allowed to be ejected out through the at least one ejecting head communicated with the compression cavity. Since the at least one ejecting head is also located on the beauty physiotherapy side of the head body, the beauty fluid or the cosmetics are ejected directly onto the skin. During use of the beauty head, there is no need to stop using the first physiotherapy piece. As long as the head body is pressed on the skin, the beauty liquid or the cosmetics are ejected directly onto the skin, realizing beauty physiotherapy while applying the beauty liquid or the cosmetics, making a use of the beauty head convenient, and bringing a good use experience.

The beauty apparatus comprises a beauty handle and the beauty head mentioned above. A mounting portion is arranged on one side of the head body opposite to the beauty physiotherapy side of the head body. The mounting portion is detachably mounted on the beauty handle.

In one embodiment, a control operating component and a second electric control piece are arranged on the beauty handle. The control operating component is configured to control an operation of the second electric control piece. A mounting groove is defined on the beauty handle. A connecting electrode is arranged on an inner wall of the mounting groove. The connecting electrode is electrically connected to the second electric control piece. A fitting electrode is arranged on the mounting portion of the beauty handle. The mounting portion is rotatably mounted in the mounting groove to enable the fitting electrode to be electrically contacted with the connecting electrode. The connecting electrode is an arc-shaped electrode or an annular electrode arranged around a rotation axis of the mounting portion.

Before using the beauty apparatus, the beauty head is mounted on the beauty handle through the mounting portion. The beauty liquid or the cosmetics applied on the skin need to be added to the liquid storage cavity. When in use, the first physiotherapy piece located on the beauty physiotherapy side of the head body is attached to the skin, and the first physiotherapy piece performs physiotherapy on the skin. When the beauty liquid or the cosmetics are needed, the beauty head is pressed on the skin through the beauty handle, so that the first physiotherapy piece directly presses against the skin. Under the reaction of the skin, the first physiotherapy piece is compressed to reduce the volume of the compression cavity. The check valve arranged in the conveying channel prevents the beauty liquid or the cosmetics from flowing into the liquid storage cavity. Therefore, the beauty liquid or the cosmetics are only allowed to be ejected out through the at least one ejecting head communicated with the compression cavity. When the first physiotherapy piece resets to increase the compression cavity, the cosmetics or the beauty liquid in the liquid storage cavity is sucked into the compression cavity. After use, the beauty head is detached from the beauty handle to facilitate cleaning of the beauty head. There is no need to stop using the beauty apparatus when applying the beauty liquid or the cosmetics on the skin. As long as the head body is pressed on the skin, the beauty liquid or the cosmetics are ejected directly onto the skin, realizing beauty physiotherapy while applying the beauty liquid or the cosmetics, making the use of the beauty head convenient, and bringing the good use experience.

### BRIEF DESCRIPTION OF DRAWINGS

The accompanying drawings that form a part of the present disclosure are used to provide a further understanding of the present disclosure. Illustrative embodiments of the present disclosure and their descriptions are used to explain the present disclosure and do not constitute an improper limitation of the present disclosure.

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

In addition, the drawings are not drawn to a 1:1 scale, and relative sizes of various components in the drawings are drawn by way of example only and not necessarily to true scale.

FIG. 1 is a structural schematic diagram of a beauty apparatus according to one embodiment of the present disclosure.

FIG. 2 is a cross-sectional schematic diagram of the beauty apparatus shown in FIG. 1.

FIG. 3 is a cross-sectional schematic diagram of a beauty head of the beauty apparatus shown in FIG. 2.

FIG. 4 is an enlarged schematic diagram of portion A shown in FIG. 3.

In the drawings:
10—beauty apparatus; 100—beauty head; 110—heade body; 111—liquid storage cavity; 112—beauty physiotherapy side; 113—compression cavity; 114—conveying channel; 115—check valve; 120—first physiotherapy piece; 130—ejecting head; 132—ejecting outlet; 134—inclined ejecting surface; 140—elastic reset piece; 150—first electric control piece; 160—second physiotherapy piece; 170—light—transmitting piece; 172—light guide column; 180—light source; 190—mounting portion; 192—fitting electrode; 200—beauty handle; 210—mounting groove; 220—control operating piece; 230—second electric control piece; 240—connecting electrode.

### DETAILED DESCRIPTION

In order to make the above objects, features, and advantages of the present disclosure clear and understood, specific embodiments of the present disclosure will be described in detail below with reference to the accompanying drawings. Specific details are set forth in the following description to fully illustrate the present disclosure. However, the present disclosure can be implemented in other ways different from that described herein, and those skilled in the art are able to do similar improvements without departing from the connotation of the present disclosure, and thus the present disclosure is not limited by the specific embodiments disclosed below.

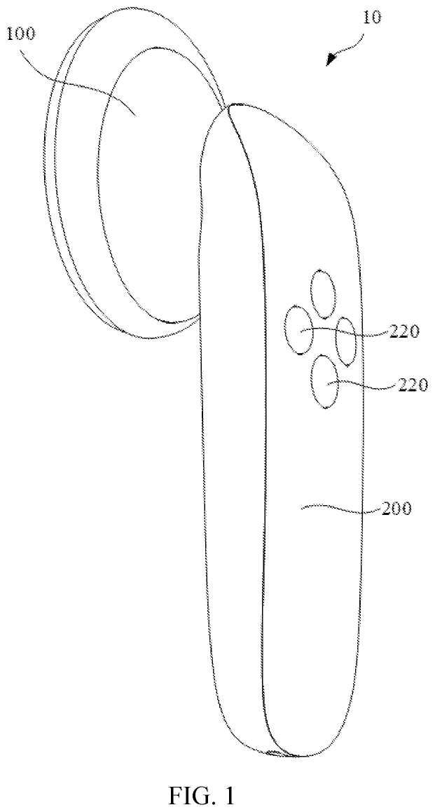

As shown in FIG. 1, a beauty apparatus 10 in one embodiment of the present disclosure realizes physiotherapy on a skin of a human body, while at least directly applying beauty liquid or cosmetics on the skin during use, making the beauty apparatus convenient to use and improving a user experience.

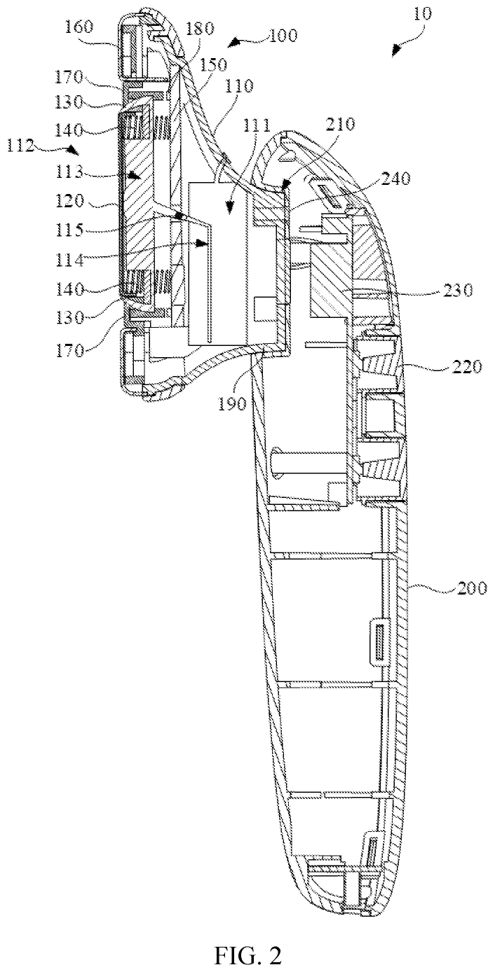

In one embodiment, as shown in FIGS. 1-3, the beauty apparatus 10 comprises a beauty head 100. The beauty head 100 comprises a head body 110, a first physiotherapy piece 120, and at least one ejecting head 130. A liquid storage cavity 111 is defined in the head body 110. One side of the head body 110 is a beauty physiotherapy side 112. The first physiotherapy piece 120 is arranged on the beauty physiotherapy side 112 of the head body 110. A compression cavity 113 is defined between the first physiotherapy piece 120 and the head body 110. The first physiotherapy piece 120 is movable relative to the head body 110 to compress or reset the compression cavity 113. The compression cavity 113 is communicated with the liquid storage cavity 111 through a conveying channel 114. A check valve 115 is arranged in the conveying channel 114. The check valve 115 is configured to control one-way flow of liquid from the liquid storage cavity 111 to the compression cavity 113. The at least one ejecting head 130 is arranged on the beauty physiotherapy side 112 of the head body 110. The compression cavity 113 is communicated with an ejecting space defined in the at least one ejecting head 130.

Before using the beauty head 100, beauty liquid or cosmetics applied on the skin of the human body need to be added to the liquid storage cavity 111. When in use, the first physiotherapy piece 120 located on the beauty physiotherapy side 112 of the head body 110 is attached to the skin, and the first physiotherapy piece 120 performs physiotherapy on the skin. When the beauty liquid or the cosmetics are needed, the beauty head 100 is pressed so that the first physiotherapy piece 120 directly presses against the skin. Under a reaction of the skin, the first physiotherapy piece 120 is compressed, so that the compression cavity 113 defined between the first physiotherapy piece 120 and the head body 110 is also compressed to discharge air in the compression cavity 113. When the first physiotherapy piece 120 resets, a volume of the compression cavity 113 increases. Since the compression cavity 113 and the liquid storage cavity 111 are communicated through the conveying channel 114, the cosmetics or the beauty liquid in the liquid storage cavity 111 is sucked into the compression cavity 113 when the volume of the compression cavity 113 increases. When the first physiotherapy piece 120 is compressed again, the compression cavity 113 is compressed and the volume of the compression cavity 113 reduces. The check valve 115 arranged in the conveying channel 114 prevents the beauty liquid or the cosmetics from flowing into the liquid storage cavity 111. Therefore, the beauty liquid or the cosmetics are only allowed to be ejected out through the at least one ejecting head 130 communicated with the compression cavity 113. Since the at least one ejecting head 130 is also located on the beauty physiotherapy side 112 of the head body 110, the beauty fluid or the cosmetics are ejected directly onto the skin.

During use of the beauty head 100, there is no need to stop using the first physiotherapy piece 120. As long as the head body 110 is pressed on the skin, the beauty liquid or the cosmetics are ejected directly onto the skin, realizing beauty physiotherapy while applying the beauty liquid or the cosmetics, making a use of the beauty head 100 convenient, and bringing a good use experience.

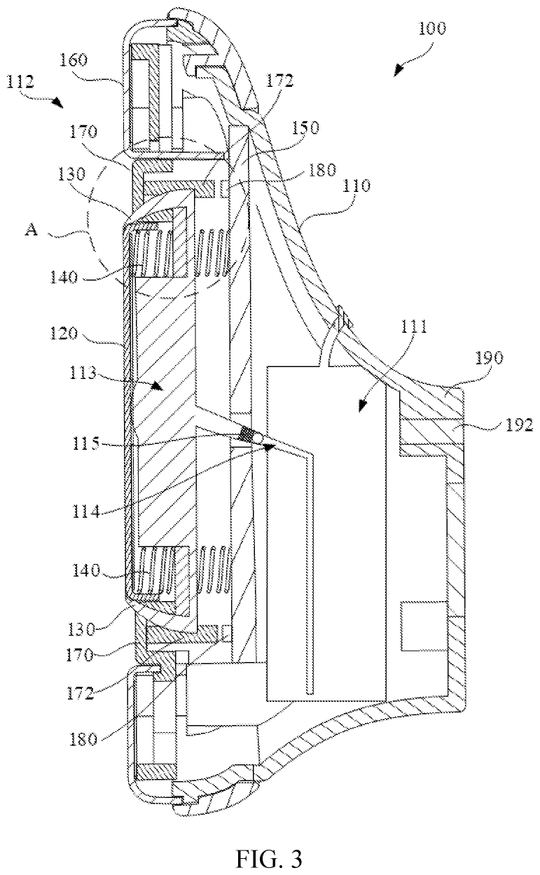

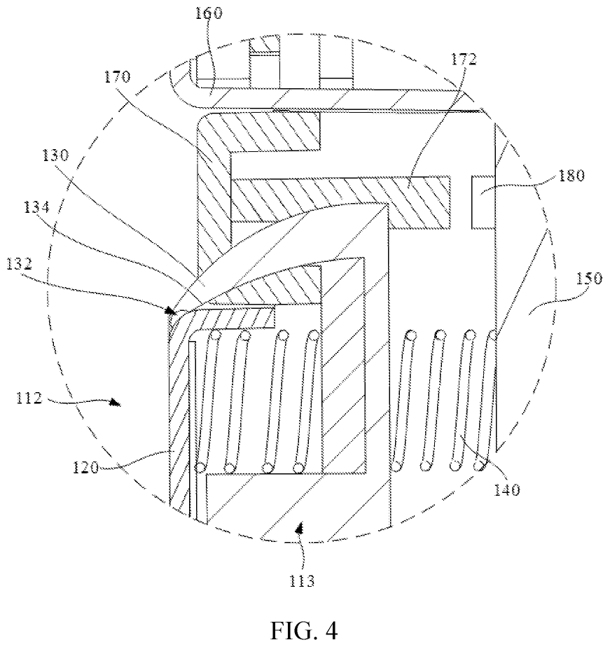

In one embodiment, as shown in FIGS. 3 and 4, a compression groove is defined on the beauty physiotherapy side 112 of the head body 110. The first physiotherapy piece 120 is mounted on the head body 110 to enable the first physiotherapy piece 120 to cover the compression groove to form the compression cavity 113. Specifically, an inner surface of the first physiotherapy piece 120 is configured as an inner wall of the compression cavity 113.

In another embodiment, an airbag capable of being compressed and reset is arranged in the compression groove. The airbag is communicated with the liquid storage cavity 111 and an ejecting channel of the at least one ejecting head 130. When the first physiotherapy piece 120 moves, the airbag is compressed or resets.

In one embodiment, the liquid storage cavity 111 is directly integrally formed in the head body 110. In another embodiment, the liquid storage cavity 111 may be a separate liquid storage tank, and the liquid storage tank is arranged inside the head body 110.

In one embodiment, as shown in FIGS. 3 and 4, the at least one ejecting head 130 defines an ejecting outlet 132 communicated with the ejecting space. The first physiotherapy piece 120 moves toward the head body 110 to compress the compression cavity 113, so that the ejecting outlet 132 is exposed outside the first physiotherapy piece 120. The first physiotherapy piece 120 is reset to reset the compression cavity 113 to cover the ejecting outlet 132. When the first physiotherapy piece 120 is compressed, the first physiotherapy piece 120 moves toward the compression cavity 113 and compresses the compression cavity 113, thereby extruding the liquid in the compression cavity 113 toward the at least one ejecting head 130. At this time, the first physiotherapy piece 120 is compressed to expose the ejecting outlet132 to facilitate the liquid to be ejected out from the ejecting outlet 132. When the first physiotherapy piece 120 is reset to cover the ejecting outlet 132, the first physiotherapy piece 120 prevents the liquid from leaking from the ejecting outlet 132 when the liquid is not needed, thereby improving reliability of the liquid being stored in the compression cavity 113 and the liquid storage cavity 111 during use.

In the embodiment, one side of the at least one ejecting head 130 facing the first physiotherapy piece 120 is an inclined ejecting surface 134. The inclined ejecting surface 134 of the at least one ejecting head 130 is inclined toward a direction away from the first physiotherapy piece 120 along a direction in which the first physiotherapy piece 120 compresses the compression cavity 113. The ejecting outlet 132 is defined on the inclined ejecting surface 134 of the at least one ejecting head 130. Since a size of the first physiotherapy piece 120 is fixed, when the first physiotherapy piece120 is reset, an outer edge of the first physiotherapy piece 120 abuts against the inclined ejecting surface 134 and thus covers the ejecting outlet 132. When the first physiotherapy piece 120 is compressed, the first physiotherapy piece120 and the inclined ejecting surface 134 are gradually separated, so the ejecting outlet 132 is exposed and the liquid is ejecting out from the ejecting outlet 132.

Specifically, the at least one ejecting head 130 may be directly formed on the head body 110. By forming the inclined ejecting surface 134 of the at least one ejecting head 130 on the head body 110, a structure of at least one ejecting head 130 is stable and reliable. In other embodiments, at least one connecting port is defined on an outer wall of the compression cavity 113, and the at least one ejecting head 130 is mounted on the head body 110. A liquid inlet end of the at least one ejecting head 130 is connected to the at least one connecting port on the outer wall of the compression cavity 113, so the compression cavity 113 is communicated with the ejecting channel of the at least one ejecting head 130.

In another embodiment, at least one elastic piece is mounted on the first physiotherapy piece 120, and one end of the at least one elastic piece is rotatably connected to the first physiotherapy piece 120 and covers on the at least one ejecting head 130. When the first physiotherapy piece120 is compressed, the at least one ejecting head 130 pushes the at least one elastic piece to expose the ejecting outlet. When the first physiotherapy piece 120 is reset, the at least one elastic piece is reset and covers the at least one ejecting head 130 again. In the embodiment, a surface where an outer end surface of the at least one ejecting head 130 is located is recessed inward toward the compression cavity 113 relative to an outer surface of the first physiotherapy piece 120.

In other embodiments, the ejecting outlet 132 of the at least one ejecting head 130 is directly defined on the outer end surface of the at least one ejecting head 130, so that when the first physiotherapy piece 120 is compressed, the liquid is ejected out directly through the at least one ejecting head 130.

In one embodiment, the first physiotherapy piece 120 is of a disc-shaped structure. The first physiotherapy piece 120 is snapped on the head body 110. The first physiotherapy piece 120 is disc-shaped, so as to define the compression cavity 113 with a certain space, thereby facilitating suction or pressing out of the liquid. Ejecting heads 130 are provided. The ejecting heads 130 are arranged around an axis of the first physiotherapy piece 120 at intervals. The ejecting space of each of the ejecting heads 130 are communicated with the compression cavity 113. By arranging the ejecting heads 130 around the first physiotherapy piece 120, the liquid is ejected out around the first physiotherapy piece 120 in a ring shape, which improves efficiency and an ejecting area of the liquid. In other embodiments, there may be only one or two ejecting heads 130.

In one embodiment, the beauty head 100 further comprises elastic reset pieces 140. The elastic reset pieces 140 are arranged on the inner side of the first physiotherapy piece 120, and the elastic reset pieces 140 reset to provide elastic forces for the first physiotherapy piece 120 to reset the compression cavity 113. When the first physiotherapy piece 120 is compressed, the elastic reset pieces 140 are compressed to store energy. When a pressure on the first physiotherapy piece 120 is released, the elastic reset pieces 140 release the stored energy to reset the first physiotherapy piece 120. The reset effect of the first physiotherapy piece 120 is improved by arranging the elastic reset pieces 140. In the embodiment, the elastic reset pieces 140 are springs. In other embodiments, the elastic reset pieces 140 may be elastic sheets, rubber bands, etc.

In one embodiment, the beauty head 100 further comprises a first electric control piece 150. The first physiotherapy piece 120 is electrically connected to the first electric control piece 150. The first electric control piece 150 is configured to control the first physiotherapy piece 120 to generate micro-current. The first physiotherapy piece 120 generates micro-current to stimulate the skin, so as to realize a purpose of beauty therapy. Specifically, the inner surface of the first physiotherapy piece 120 is configured as the inner wall of the compression cavity 113, so that the liquid in the compression cavity 113 is electrified through the first physical physiotherapy piece120, so that the liquid with micro-current is ejected through the ejecting heads 130. The liquid with micro-current improves the physiotherapy and beauty effect on the skin.

Specifically, the first electric control piece 150 is located between the compression cavity 113 and the liquid storage cavity 111, and a passing hole is defined on the first electric control piece 150 for the conveying channel 114 to pass through. In other embodiments, the first electric control piece 150 is arranged at other positions within the head body 110, as long as it does not affect an electrical connection between the first physiotherapy piece 120 and the first electric control piece 150, and does not affect a communication between the compression cavity 113 and the liquid storage cavity 111 through the conveying channel 114.

In the embodiment, the first physiotherapy piece 120 is electrically connected to the first electric control piece 150 through the elastic reset pieces 140. Specifically, the elastic reset pieces 140 are conductive springs. The elastic reset pieces 140 not only provide the elastic piece for the first physiotherapy piece 120 to rest, but also enables the electrical connection between the first physiotherapy piece 140 and the first electric control piece 150. In other embodiments, the first physiotherapy piece 120 may also be directly electrically connected to the first electric control piece 150 through wires.

In one embodiment, the beauty head 100 further comprises a second physiotherapy piece 160. The second physiotherapy piece 160 is mounted on the beauty physiotherapy side 112 of the head body 110. The second physiotherapy piece 160 is electrically connected to the first electric control piece 150. The first electric control piece 150 is configured to control the first physiotherapy piece 120 and the second physiotherapy piece 160 to generate micro-current. The ejecting heads 130 are located between the first physiotherapy piece 120 and the second physiotherapy piece 160. By arranging the second physiotherapy piece 160, a micro-current generation area is increased and the physiotherapy effect is improved.

Specifically, the second physiotherapy piece160 is of an annular structure. The first physiotherapy piece 120 is located within an inner ring of the second physiotherapy piece 160. The ejecting heads 130 are located between the first physiotherapy piece 120 and the second physiotherapy piece 160. In other embodiments, a plurality of second physiotherapy pieces 160 are provided, and the plurality of second physiotherapy pieces 160 are distributed at intervals on the beauty physiotherapy side 112 of the head body 110, as long as the micro-current configured to stimulate the skin is generated by the second physiotherapy pieces 160 and the first physiotherapy piece 120.

In one embodiment, the beauty head 100 further comprises a heating piece. The heating piece is attached to the first physiotherapy piece 120. The heating piece is electrically connected to the first electric control piece 150. The first electric control piece 150 is configured to control a heating temperature of the heating piece. By controlling the heating of the first physiotherapy piece 120 by the heating piece, heating physiotherapy on the skin is further realized, which improves the cosmetic effect.

In another embodiment, heating pieces are arranged on the first physiotherapy piece 120 and the second physiotherapy piece160, and the heating pieces are configured to heat the first physiotherapy piece 120 and the second physiotherapy piece160.

In one embodiment, the beauty head 100 further comprises a light-transmitting piece 170 and a light source 180. The light source 180 is arranged on the first electric control piece 150. The light-transmitting piece 170 is arranged on the beauty physiotherapy side 112 of the head body 110 and faces the light source 180. An outer surface of the light-transmitting piece 170 is concave in a direction facing the compression cavity 113 relative to the outer surface of the first physiotherapy piece 120. The light-transmitting piece 170 is an annular piece. The first physiotherapy piece 120 is located in an inner ring of the light-transmitting piece 170. A limiting space is defined on a side of the light-transmitting piece 170 facing the first physiotherapy piece 120. The ejecting heads 130 are arranged in the limiting space. The light source 180 is configured to emit infrared light or blue light that beautifies the skin, which further increases the function of the beauty head 100, realizing spectrum physiotherapy on the skin during a process of micro-current beauty physiotherapy.

In the embodiment, the light-transmitting piece 170 is located between the first physiotherapy piece 120 and the second physiotherapy piece 160, and the first physiotherapy piece120 and the second physiotherapy piece160 are spaced apart by the light-transmitting piece 170.

As shown in FIG. 3, in one embodiment, the first electric control piece 150 is located between the compression cavity 113 and the liquid storage cavity 111. Specifically, a light guide column 172 is arranged on one side of the light-transmitting piece 170 facing the light source 180. One end of the light guide column 172 away from the light-transmitting piece 170 is aligned with the light source 180. Since the light source 180 is arranged on the first electric control piece 150, the light guide column 172 is guides the infrared light or the blue light from the light source 180 to the light-transmitting piece 170, so that the infrared light or the blue light emitted by the light source 180 is effectively emitted out through the light-transmitting piece 170.

As shown in FIGS. 1-3, in one embodiment, the beauty apparatus 10 further comprises a beauty handle 200. A mounting portion 190 is arranged on one side of the head body 110 opposite to the beauty physiotherapy side 112 of the head body 110. The mounting portion 190 is detachably mounted on the beauty handle 200.

Before using the beauty apparatus 10, the beauty head 100 is mounted on the beauty handle 200 through the mounting portion 190. The beauty liquid or the cosmetics applied on the skin need to be added to the liquid storage cavity 111. When in use, the first physiotherapy piece 120 located on the beauty physiotherapy side 112 of the head body 110 is attached to the skin, and the first physiotherapy piece 120 performs physiotherapy on the skin. When the beauty liquid or the cosmetics are needed, the beauty head 100 is pressed on the skin through the beauty handle, so that the first physiotherapy piece 120 directly presses against the skin. Under the reaction of the skin, the first physiotherapy piece 120 is compressed to reduce the volume of the compression cavity 113. The check valve 115 arranged in the conveying channel 114 prevents the beauty liquid or the cosmetics from flowing into the liquid storage cavity 111. Therefore, the beauty liquid or the cosmetics are only allowed to be ejected out through the ejecting heads 130 communicated with the compression cavity 113. When the first physiotherapy piece 120 resets to increase the compression cavity 113, the cosmetics or the beauty liquid in the liquid storage cavity 111 is sucked into the compression cavity 113. After use, the beauty head 100 is detached from the beauty handle to facilitate cleaning of the beauty head 100. There is no need to stop using the beauty apparatus when applying the beauty liquid or the cosmetics on the skin. As long as the head body 110 is pressed on the skin, the beauty liquid or the cosmetics are ejected directly onto the skin, realizing beauty physiotherapy while applying the beauty liquid or the cosmetics, making the use of the beauty head 100 convenient, and bringing the good use experience.

In one embodiment, a mounting groove 210 is defined on the beauty handle 200. The mounting portion 190 is detachably mounted in the mounting groove 210.

Specifically, an elastic fastener may be arranged on an inner wall of the mounting groove 210. After the mounting portion 190 is mounted in the mounting groove 210, the mounting portion 190 is fastened to the mounting groove 210 through the elastic fastener. In other embodiments, a limiting convex ring may be arranged on the inner wall of the mounting groove 210, and a limiting groove is defined on the mounting portion 190. The limiting convex ring is inserted into and engaged with the limiting groove. Alternatively, the beauty head 100 may also be mounted on the beauty handle 200 in other ways, as long as the beauty head 100 is detachable connected to the beauty handle 200.

In one embodiment, a control operating component 220 and a second electric control piece 230 are arranged on the beauty handle 200. The control operating component 220 is configured to control an operation of the second electric control piece 230. A connecting electrode 240 is arranged on an inner wall of the mounting groove. The connecting electrode 240 is electrically connected to the second electric control piece 230. A fitting electrode 192 is arranged on the mounting portion 190 of the beauty handle 200. The mounting portion 190 is detachably mounted in the mounting groove 210 to enable the fitting electrode 192 to be electrically contacted with the connecting electrode 240.

By arranging the connecting electrode 240 and the fitting electrode 192, it is convenient for the second electric control piece 230 to be electrically connected to the first electric control piece 150 through the connecting electrode 240 and the fitting electrode 192, thereby enabling the control of the second electric control piece 230 by the control operating component 220 on one side of the beauty handle 200.

In the embodiment, the control operating component 220 are buttons arranged on the beauty handle 200. By operating the buttons, the second electric control piece 230 is allowed to be controlled by the first electric control piece 150. In other embodiments, the control operating component 220 may be a touch display, and the first electric control piece 150 is controlled by touch operation performed on the touch display.

In one embodiment, the mounting portion 190 is rotatably mounted in the mounting groove 210, so that the fitting electrode 192 contacts the connecting electrode 240. The connecting electrode 240 is an arc-shaped electrode or an annular electrode arranged around a rotation axis of the mounting portion 190. The mounting portion 190 is rotatable in the mounting groove 210, so that a position or an angle of the beauty physiotherapy side 112 of the head body 110 is adjustable, which improves a convenience of using the beauty head 100. Therefore, the head body is allowed to be attached to different positions of the skin of the user. The connecting electrode 240 the arc-shaped electrode or the annular electrode arranged around the rotation axis of the mounting portion 190, which facilitates stable contact between the fitting electrode 192 and the connecting electrode 240 during the rotation of the mounting portion 190, ensuring electrical connection between the fitting electrode 192 and the connecting electrode 240 during the rotation.

The beauty apparatus 10 not only realizes beauty physiotherapy with micro-current effect, but also realizes spectrum beauty. Since the first physiotherapy piece 120 is capable of being compressed, automatic ejection of the beauty liquid is realized, thereby improving the use convenience. Moreover, since an inner side of the second physiotherapy piece160 is configured as an inner wall of the compression cavity 113, the ejected beauty liquid carries the micro-current, which further improves the beauty physiotherapy effect.

It should be understood that in the description of the present disclosure terms such as “central”, “lateral”, “lengthways”, “length”, “width”, “thickness”, “upper”, “lower”, “left”, “right”, “vertical”, “horizontal”, “top”, “bottom”, “inner”, “outer”, “clockwise”, “counterclockwise”, etc., indicate direction or position relationships shown based on the drawings, and are only intended to facilitate the description of the present disclosure and the simplification of the description rather than to indicate or imply that the indicated device or element must have a specific direction or constructed and operated in a specific direction, and therefore, shall not be understood as a limitation to the present disclosure.

In addition, terms such as “first” and “second” are only used for the purpose of description, rather than being understood to indicate or imply relative importance or hint the number of indicated technical features. Thus, the feature limited by “first” and “second” can explicitly or impliedly include one or more features. In the description of the present disclosure, the meaning of “a plurality of” is two or more unless otherwise specified.

It should be noted in the description of the present disclosure that, unless otherwise regulated and defined, terms such as “installation,” “bonded,” and “connection” shall be understood in broad sense, and for example, may refer to fixed connection or detachable connection or integral connection; may refer to mechanical connection or electrical connection; and may refer to direct connection or indirect connection through an intermediate medium or inner communication of two elements. For those of ordinary skill in the art, the meanings of the above terms in the present disclosure may be understood according to concrete conditions.

In the present disclosure, unless expressly stipulated and defined otherwise, the first feature is arranged “above” or “below” the second feature may means that the first feature directly contact the second feature, or the first feature does not directly contact the second feature but connected to the second feature through other features between them. Furthermore, the first feature is arranged “on”, “above” or “over” the second feature means that the first feature may arrange directly above and obliquely above the second feature, or it may merely indicate that a level of the first feature is greater than a level of the second feature. The first feature is arranged “below”, “under”, and “beneath” the second feature means that the first feature is arranged directly below and obliquely below the second feature, or it simply means that the level of the first feature is less than the level of second feature.

It should be noted that when a component is referred to as being “fixed to” or “arranged on” another component, it can be directly fixed on the other component or it may be indirectly fixed on the other component through a mediation component. When a component is considered to be “connected” to another component, it can be directly connected to the other component or it may be indirectly connected to the other component through a mediation component. When a component is considered to be “arranged on” another component, it may be directly arranged on the other component or it may be arranged on the other component through a mediation component. The terms “vertical”, “horizontal”, “left”, “right”, and similar expressions are used herein for illustrative purposes only.

The above-mentioned embodiments only represent some embodiments of the present disclosure. The descriptions thereof are specific and detailed, but should not be construed as a limitation of the scope of the present disclosure. It should be pointed out that for those of ordinary skill in the art, without departing from the concept of the present disclosure, modifications and improvements can be made. The modifications and the improvements belong to the protection scope of the present disclosure. Therefore, the protection scope of the present disclosure should be subject to the attached claims.

What is claimed is:

1. A beauty head, comprising:
a head body,
a first physiotherapy piece, and
at least one ejecting head;
wherein a liquid storage cavity is defined in the head body; one side of the head body is a beauty physiotherapy side; the first physiotherapy piece is arranged on the beauty physiotherapy side of the head body; a compression cavity is defined between the first physiotherapy piece and the head body; the first physiotherapy piece is movable relative to the head body to compress or reset the compression cavity; the compression cavity is communicated with the liquid storage cavity through a conveying channel; a check valve is arranged in the conveying channel; the check valve is configured to control one-way flow of liquid from the liquid storage cavity to the compression cavity;
the at least one ejecting head is arranged on the beauty physiotherapy side of the head body; the compression cavity is communicated with an ejecting space defined in the at least one ejecting head.

2. The beauty head according to claim 1, wherein the at least one ejecting head defines an ejecting outlet communicated with the ejecting space; the first physiotherapy piece moves toward the head body to compress the compression cavity, so that the ejecting outlet is exposed outside the first physiotherapy piece; the first physiotherapy piece is reset to reset the compression cavity to cover the ejecting outlet.

3. The beauty head according to claim 2, wherein one side of the at least one ejecting head facing the first physiotherapy piece is an inclined ejecting surface; the inclined ejecting surface of the at least one ejecting head is inclined toward a direction away from the first physiotherapy piece along a direction in which the first physiotherapy piece compresses the compression cavity; the ejecting outlet is defined on the inclined ejecting surface of the at least one ejecting head.

4. The beauty head according to claim 1, wherein the first physiotherapy piece is of a disc-shaped structure; the first physiotherapy piece is snapped on the head body; ejecting heads are provided; the ejecting heads are arranged around an axis of the first physiotherapy piece at intervals; the ejecting space of each of the ejecting heads are communicated with the compression cavity.

5. The beauty head according to claim 1, wherein the beauty head further comprises a first electric control piece; the first physiotherapy piece is electrically connected to the first electric control piece; the first electric control piece is configured to control the first physiotherapy piece to generate micro-current; a compression groove is defined on the beauty physiotherapy side of the head body; the first physiotherapy piece is mounted on the head body to enable the first physiotherapy piece to cover the compression groove to form the compression cavity; an inner surface of the first physiotherapy piece is configured as an inner wall of the compression cavity.

6. The beauty head according to claim 5, wherein the beauty head further comprises elastic reset pieces; the elastic reset pieces are arranged on the inner side of the first physiotherapy piece, and the elastic reset pieces reset to provide elastic forces for the first physiotherapy piece to reset the compression cavity; the first physiotherapy piece is electrically connected to the first electric control piece through the elastic reset pieces.

7. The beauty head according to claim 5, wherein the beauty head further comprises a heating piece; the heating piece is attached to the first physiotherapy piece; the heating piece is electrically connected to the first electric control piece; the first electric control piece is configured to control a heating temperature of the heating piece.

8. The beauty head according to claim 5, wherein the beauty head further comprises a light-transmitting piece and a light source; the light source is arranged on the first electric control piece; the light-transmitting piece is arranged on the beauty physiotherapy side of the head body and faces the light source; an outer surface of the light-transmitting piece is concave in a direction facing the compression cavity relative to the outer surface of the first physiotherapy piece; the light-transmitting piece is an annular piece; the first physiotherapy piece is located in an inner ring of the light-transmitting piece; a limiting space is defined on a side of the light-transmitting piece facing the first physiotherapy piece; the at least one ejecting head is arranged in the limiting space.

9. A beauty apparatus, comprising:
a beauty handle, and
the beauty head according to claim 1;
wherein a mounting portion is arranged on one side of the head body opposite to the beauty physiotherapy side of the head body; the mounting portion is detachably mounted on the beauty handle.

10. The beauty apparatus according to claim 9, wherein a control operating component and a second electric control piece are arranged on the beauty handle; the control operating component is configured to control an operation of the second electric control piece; a mounting groove is defined on the beauty handle; a connecting electrode is arranged on an inner wall of the mounting groove; the connecting electrode is electrically connected to the second electric control piece; a fitting electrode is arranged on the mounting portion of the beauty handle; the mounting portion is rotatably mounted in the mounting groove to enable the fitting electrode to be electrically contacted with the connecting electrode; the connecting electrode is an arc-shaped electrode or an annular electrode arranged around a rotation axis of the mounting portion.
